(19) United States
(12) Patent Application Publication
(10) Pub. No.: US 2019/0167512 A1
(43) Pub. Date: Jun. 6, 2019

(54) Automated and Interactive Massage Therapy System and Method

(71) Applicant: Interactive Massage Therapy, LLC, Provo, UT (US)

(72) Inventors: Clayton Forsythe, Provo, UT (US); Dillon Forsythe, Provo, UT (US)

(21) Appl. No.: 16/209,567

(22) Filed: Dec. 4, 2018

Related U.S. Application Data
(60) Provisional application No. 62/594,166, filed on Dec. 4, 2017.

(51) Int. Cl. A61H 15/00; G16H 20/30; A61B 34/30

(52) U.S. Cl. CPC A61H 15/0078; A61H 7/004; A61B 34/30; G16H 20/30

(57) ABSTRACT

A robotic system for massage therapy includes a selectively movable robotic arm having a contact surface and a sensor associated with the selectively movable robotic arm. The sensor detects an amount of tactile pressure exerted upon the contact surface. The robotic system can additionally include a computer system configured to activate the selectively movable robotic arm and execute a massage therapy protocol using the robotic arm and sensor.

### CROSS-REFERENCE TO RELATED APPLICATIONS

This application claims the benefit of and priority to U.S. Provisional Patent Application Ser. No. 62/594,166 filed on Dec. 4, 2017 and entitled “Automated and Interactive Massage Therapy System and Method,” and which application is expressly incorporated herein by reference in its entirety.

### BACKGROUND

### Technical Field

This disclosure generally relates to systems configured to manipulate the human body. More specifically, the present disclosure relates to automated and interactive robotic systems implementing massage therapy.

### Related Technology

The human body is a complex network of skeletal and muscular components that work together to provide the structural and functional properties required for human mobility, flexibility, and agility. Unsurprisingly, many health issues stem from the misalignment, fatigue, or impaired mobility of bones and/or muscles within this complex network. Professionals, such as chiropractors and doctors of osteopathic medicine, are trained to identify and correct pathological/problematic skeletal misalignments through physical manipulation techniques. These can include, for example, stretching, applying gentle pressure, and/or resistance to joints and muscles. Through physical manipulation of the musculoskeletal system, these practitioners can reduce or alleviate pain, prevent additional trauma, or promote the general health and well-being of their patients.

Physical manipulation is often only the first step in a treatment regimen, however. Patients are often prescribed massage therapy following manipulation, and this can serve myriad purposes. In some instances, massage therapy is prescribed to rehabilitate the injury or reduce pain or soreness associated with the injury or resulting from the manipulation, itself. Massage therapy can additionally be prescribed to relax muscles or to reduce stress and anxiety, which in some instances can be part of the cause of the patient's injury. A tight hamstring, for example, could be causing lower back pain, and by relaxing the hamstring through massage therapy, the back pain can be alleviated or reduced.

Problematically, patients often fail to receive the prescribed treatment, which can include one or more of a failure to provide the prescribed massage modality at the prescribed location and/or a failure to consistently provide the prescribed treatment for the prescribed duration (e.g., in a single session or multiple sessions). Often, the patient fails to receive the prescribed treatment due to unclear instructions or because of a miscommunication between the prescribing healthcare provider and the massage therapist, but in some instances, the patient does not receive the prescribed treatment because the massage therapist fails to implement a properly articulated prescription—whether purposely or inadvertently.

In other instances, the prescribing healthcare provider opts for a mechanical massage system in lieu of a massage therapist. Current massage systems, however, are fraught with inefficiencies and are generally incapable of providing the optimal modality or precision necessary to implement optimal treatment regimens. For example, current massage systems are incapable of performing site-specific, personalized treatments in accordance with a prescribed regimen. Instead, these systems perform a constant, repetitive motion that fails to specifically or directly address the problem area.

Whether the failed implementation of the prescribed treatment is the result of incompetence (e.g., stemming from unclear instructions, a miscommunication, or a difference of opinion as to the proper treatment) or the result of unfit mechanical systems, the outcome is the same: the patient fails to receive the prescribed treatment. When a patient fails to receive the treatments prescribed by their healthcare provider, the patient's condition can worsen, their recovery time can be extended, and overall, the patient fails to receive quality, consistent care.

Accordingly, there are a number of disadvantages with massage therapy systems that can be addressed.

### BRIEF SUMMARY

Implementations of the present disclosure solve one or more of the foregoing or other problems in the art with massage therapy systems. In particular, one or more implementations can include a robotic system for massage therapy that includes a selectively movable robotic arm having a contact surface and a sensor associated with the selectively movable robotic arm. The sensor detects an amount of tactile pressure exerted upon the contact surface.

Systems of the present disclosure can also include an imaging device, which in some embodiments is movable and configured to capture a three-dimensional image of a portion of a person receiving massage therapy

Systems of the present disclosure can additionally, or alternatively, include a computer system associated with one or more of the selectively movable robotic arm and the senor. The computer system can include processor(s) and hardware storage device(s) having stored thereon computer-executable instructions that when executed by the processor(s) cause the computer system to at least (i) receive image data from an imaging device and (ii) identify a therapeutic site based on the image data. The computer-executable instructions can additionally cause the computer system to position the contact surface of the selectively movable robotic arm at or near the therapeutic site, receive a massage therapy protocol, activate the selectively movable arm to perform the massage therapy protocol, receive tactile pressure data from the sensor during therapy, modify a position of the selectively movable arm to increase or decrease the amount of tactile pressure exerted upon the contact surface in accordance with the massage therapy protocol or in accordance with a maximum tactile pressure threshold, and/or receive command(s) from a microphone and/or a user interface communicatively coupled to the computer system, adjust the massage therapy protocol based on the received command(s).

One or more implementations can include a computer system for massage therapy having processor(s) and computer-readable hardware storage device(s) having stored thereon computer-executable instructions that when executed by the processor(s) cause the computer system to at least (i) receive a massage therapy protocol, (ii) receive image data from an imaging device in electrical communication with the computer system, (iii) based on the image data and the massage therapy protocol, identify a therapeutic location, and (iv) activate a selectively movable arm in electrical communication with the computer system to execute at least a portion of the massage therapy protocol. The selectively movable arm has a contact surface and can be associated with a sensor for detecting an amount of tactile pressure exerted upon the contact surface.

One or more implementations include a computer-implemented method for automated massage therapy. The method includes (i) receiving a massage therapy protocol, (ii) activating a selectively movable arm—the selectively movable arm having a contact surface and being associated with a sensor for detecting an amount of tactile pressure exerted upon the contact surface—and (iii) executing the massage therapy protocol with the activated arm.

Accordingly, systems and methods for automated massage therapy are disclosed.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims, or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 illustrates an exemplary computer system;

FIG. 2 illustrates an exemplary system for automated massage therapy according to one or more embodiments of the present disclosure;

FIG. 3 illustrates another exemplary system for automated massage therapy according to one or more embodiments of the present disclosure; and

FIG. 4 illustrates an exemplary method of automated massage therapy.

### DETAILED DESCRIPTION

Before describing various embodiments of the present disclosure in detail, it is to be understood that this disclosure is not limited to the parameters of the particularly exemplified systems, methods, apparatus, products, and/or processes, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments, and is not necessarily intended to limit the scope of the claimed invention.

As discussed above, patients prescribed massage therapy often fail to receive their prescribed regimen, which can include one or more of a failure by a healthcare provider to administer the prescribed massage modality at the prescribed therapeutic location and/or a failure by a healthcare provider to consistently administer the prescribed treatment for the prescribed duration (e.g., in a single session or multiple sessions). Sometimes the patient fails to receive the prescribed treatment due to unclear instructions between healthcare providers (e.g., between the prescribing chiropractor and the massage therapist) or because of a miscommunication between the prescribing healthcare provider and the massage therapist. In some instances, the patient does not receive the prescribed treatment because the massage therapist fails to implement a properly articulated prescription—whether purposely or inadvertently.

In other instances, the patient fails to receive adequate or optimal treatment because the prescribing healthcare provider opts for a mechanical massage system in lieu of a massage therapist. Current massage systems are fraught with inefficiencies and are generally incapable of providing the optimal modality or precision necessary to implement optimal treatment regimens. For example, current massage systems are incapable of performing site-specific, personalized treatments in accordance with a prescribed regimen. Instead, these systems perform a constant, repetitive motion that fails to specifically or directly address the problem area.

Whether the failed implementation of the prescribed treatment is the result of incompetence (e.g., stemming from unclear instructions, a miscommunication, or a difference of opinion as to the proper treatment) or the result of unfit mechanical systems, the outcome is the same: the patient fails to receive the prescribed treatment. When a patient fails to receive the treatments prescribed by their healthcare provider, the patient's condition can worsen, their recovery time can be extended, and overall, the patient fails to receive quality, consistent care.

Systems and methods disclosed herein provide solutions to the aforementioned problems in the art of massage therapy. For example, the automated massage therapy systems disclosed herein enable a prescribed massage therapy to be administered to a designated therapeutic site. A combination of sensors and imaging devices can be used to ensure the prescribed pressure is administered to the prescribed therapeutic site, and in some embodiments, the automated system is interactive, responding to patient commands. These features enable the disclosed systems to safely administer a wide range of prescribed massage modalities to a particular therapeutic site and to dynamically respond to a patient's preferences (e.g., more or less pressure).

In some embodiments, the disclosed systems can be substituted with a massage therapist without a substantial reduction in therapeutic benefits. The imaging devices associated with some of the disclosed massage systems can identify the patient and direct movable robotic arms to the prescribed therapeutic site. Using one or more sensors that detect tactile pressure exerted on a contact surface of the robotic arm, the disclosed massage systems further enable precise and consistent therapy. This can improve the consistency and quality of therapy provided to patients and can facilitate improvement of the patient's condition, reduce their recovery time, and improve the patient's satisfaction and quality of care—particularly when compared with the ineffective prior art massage system or even when compared with the variability and inconsistency provided by massage therapists.

In some instances, the massage therapy system includes one or more computer system. The computer system may implement one or more methods to enable massage therapy, including, for example, identification of a therapeutic site, directing and monitoring one or more robotic arms during execution of a massage therapy protocol, and receiving commands from a patient (and making any patient requested adjustments to the executed massage therapy protocol). It will be appreciated that computing systems are increasingly taking a wide variety of forms. Computing systems may, for example, be handheld devices, appliances, laptop computers, desktop computers, mainframes, distributed computing systems, datacenters, or even devices that have not conventionally been considered a computing system, such as wearables (e.g., glasses). In this description and in the claims, the term “computer system” or “computing system” is defined broadly as including any device or system—or combination thereof—that includes at least one physical and tangible processor and a physical and tangible memory capable of having thereon computer-executable instructions that may be executed by a processor. The memory may take any form and may depend on the nature and form of the computing system. A computing system may be distributed over a network environment and may include multiple constituent computing systems.

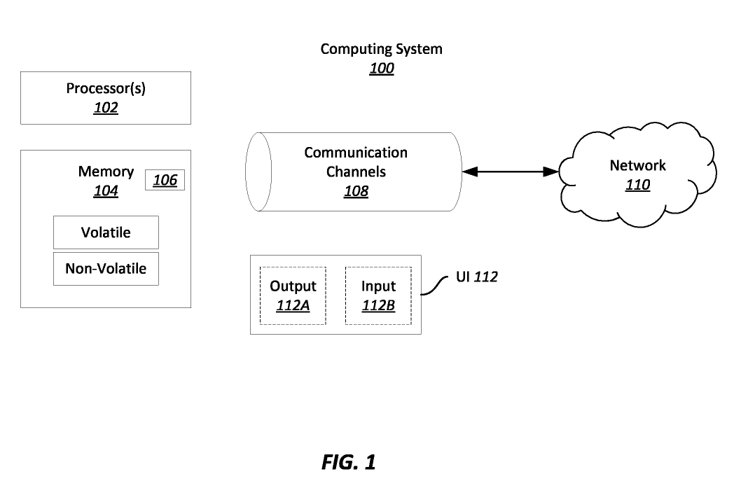

As illustrated in FIG. 1, in its most basic configuration, a computing system 100 typically includes at least one hardware processing unit 102 and memory 104. The memory 104 may be physical system memory, which may be volatile, non-volatile, or some combination of the two. The term “memory” may also be used herein to refer to non-volatile mass storage such as physical storage media. The computing system 100 may be a standalone or distributed system. If the computing system is distributed, the processing, memory, and/or storage capability may be distributed as well.

Any number and/or type of general purpose or special purpose computing systems described above can be configured to predict and/or modify resource utilization and demands. For example, the database(s) may be stored in the memory 104 of computing system 100, and for the purpose of this disclosure, any general purpose or special purpose computer storing at least a portion of one or more databases will be generally referred to as a database server. It should be appreciated, however, that the term “database server” as used herein should also be understood to include the back-end system of a database application that performs tasks such as data analysis, storage, data manipulation, archiving, and other non-user specific tasks.

The computing system 100 also has thereon multiple structures often referred to as an “executable component.” For instance, the memory 104 of the computing system 100 is illustrated as including executable component 106. The term “executable component” is the name for a structure that is well understood to one of ordinary skill in the art in the field of computing as being a structure that can be software, hardware, or a combination thereof. For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media.

The structure of the executable component exists on a computer-readable medium in such a form that it is operable, when executed by one or more processors of the computing system, to cause the computing system to perform one or more function, such as the functions and methods described herein. Such a structure may be computer-readable directly by the processors—as is the case if the executable component were binary. Alternatively, the structure may be structured to be interpretable and/or compiled—whether in a single stage or in multiple stages—so as to generate such binary that is directly interpretable by the processors. Such an understanding of exemplary structures of an executable component is well within the understanding of one of ordinary skill in the art of computing when using the term “executable component.”

The term “executable component” is also well understood by one of ordinary skill as including structures that are implemented exclusively or near-exclusively in hardware, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination. In this description, the terms “component,” “service,” “engine,” “module,” “control,” “generator,” or the like may also be used. As used in this description and in this case, these terms—whether expressed with or without a modifying clause—are also intended to be synonymous with the term “executable component,” and thus also have a structure that is well understood by those of ordinary skill in the art of computing.

In the description that follows, embodiments are described with reference to acts that are performed by one or more computing systems. If such acts are implemented in software, one or more processors (of the associated computing system that performs the act) direct the operation of the computing system in response to having executed computer-executable instructions that constitute an executable component. For example, such computer-executable instructions may be embodied on one or more computer-readable media that form a computer program product. An example of such an operation involves the manipulation of data.

The computer-executable instructions (and the manipulated data) may be stored in the memory 104 of the computing system 100. Computing system 100 may also contain communication channels 108 that allow the computing system 100 to communicate with other computing systems over, for example, network 110.

While not all computing systems require a user interface, in some embodiments the computing system 100 includes a user interface 112 for use in interfacing with a user. The user interface 112 may include output mechanisms 112A as well as input mechanisms 112B. The principles described herein are not limited to the precise output mechanisms 112A or input mechanisms 112B as such will depend on the nature of the device. However, output mechanisms 112A might include, for instance, speakers, displays, tactile output, holograms, and so forth. Examples of input mechanisms 112B might include, for instance, microphones, touchscreens, holograms, cameras, keyboards, mouse, or other pointer input, sensors of any type, and so forth.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example—not limitation—embodiments of the disclosure can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium which can be used to store desired program code in the form of computer-executable instructions or data structures and which can be accessed and executed by a general purpose or special purpose computing system to implement the disclosed functionality of the disclosure.

Transmission media can include a network and/or data links which can be used to carry desired program code in the form of computer-executable instructions or data structures and which can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Although the subject matter described herein is provided in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the concepts of this disclosure may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, tablets, mobile telephones, PDAs, pagers, routers, switches, datacenters, wearables (e.g., glasses) and the like. The invention may also be practiced in distributed system environments where local and remote computing systems, which are linked (either by hardwired data links, wireless data links, or by a combination of hardwired and wireless data links) through a network, both perform tasks. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

### Massage Systems

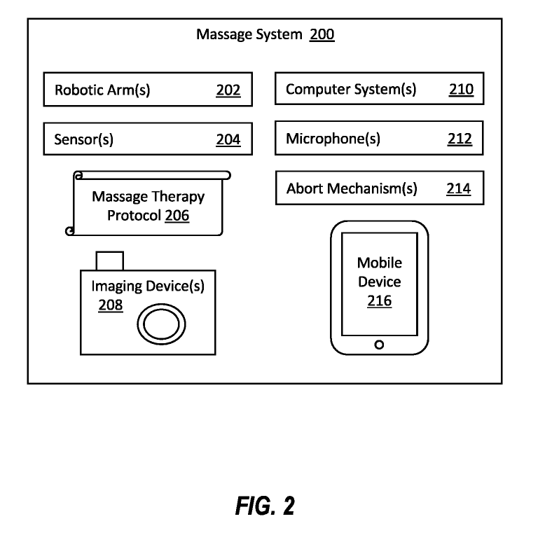

Referring now to FIG. 2, an exemplary massage system 200 is illustrated. The massage system 200 includes one or more robotic arms 202 and one or more sensors 204 associated with the robotic arms 202. In some embodiments, the robotic arms 202 can be selectively movable and include a distal end for contacting the patient and delivering massage therapy at a designated therapeutic site. Each robotic arm 202 can include one or a plurality of distal ends with each distal end having a contact surface. The massage therapy is delivered through the contact surface and is associated with one or more sensors 204 that detect an amount of tactile pressure exerted on the contact surface. The sensors 204 allow precise amounts of tactile pressure to be administered through the robotic arm 202.

The massage system 200 additionally includes a massage therapy protocol 206, which provides, for example, a location of the therapeutic site, a massage therapy modality to apply at the therapeutic site (e.g., movement sequences and combinations of tactile pressure), a duration of therapy, and/or one or more amounts of tactile pressure to apply during therapy.

The massage system 200 additionally includes one or more imaging devices 208. The imaging devices 208 capture images or video of at least a portion of the patient's body, those images or video can be used together with information obtained from the massage therapy protocol 206 to identify a therapeutic site on the patient. Additionally, or alternatively, the imaging devices 208 can be used to guide the robotic arms 202 to the therapeutic site and/or direct the movement of the robotic arms 202 through the designated movements prescribed within the massage therapy protocol 206. The sensors 204 can ensure the prescribed amount of tactile pressure is delivered by the robotic arms in accordance with the massage therapy protocol 206.

The sensors 204 and/or the imaging devices 208 can also prevent the robotic arm from applying excessive tactile pressure to a patient. In some embodiments, the robotic arms 202 are configured to deliver no more than a threshold amount of tactile pressure (e.g., 20 psi, 10 psi, 5 psi, or less, preferably 10 psi or less), and the sensors 204 can monitor the amount of tactile pressure exerted upon the contact surface of each robotic arm 202 to reduce the amount of tactile pressure if it approaches or meets the threshold amount of tactile pressure. Additionally, or alternatively, the imaging devices 208 can move and gather three-dimensional image data. This three-dimensional image data can be used to ensure the contact surface of the robotic arms 202 does not press into the patient beyond a threshold depth (e.g., 2 inches, 1.5 inches, 1 inch, 0.75 inches, 0.5 inches, 0.25 inches, or less, preferably 1 inch or less).

Any of the robotic arms 202, sensors 204, or imaging devices 208 can be in electrical communication with one or more computer systems 210. The computer system 210 can receive information from the massage therapy protocol 206, the sensors 204 and/or the imaging devices 208 to control the robotic arms 202. This can include, for example, identifying a therapeutic site on the patient using image data obtained from the imaging devices 208 in combination with the therapeutic site prescribed by the massage therapy protocol 206, directing the robotic arms 202 to the therapeutic site, and moving the robotic arms 202 to deliver the therapy prescribed within the massage therapy protocol 206. The computer system to 10 can additionally monitor information from the sensors 204 to ensure the prescribed amount of pressure is delivered to the patient by the robotic arms 202.

The massage system 200 can additionally include one or more microphones 212 in electrical communication with the computer system 210. The microphones 212 can receive one or more verbal patient commands, which when received by the computer system 210, can augment one or more operating properties of the massage system 200. For example, a patient can request deeper or lighter pressure, and the computing system 210 can adjust the massage therapy protocol 206 to accommodate the changed pressure requested by the patient. As an additional example, the microphone 212 can receive a verbal command to extend therapy for a longer duration of time or to immediately cease operation of the massage system 200. The latter verbal command is, in some embodiments, an abort mechanism 214.

The massage system 200 can include one or more abort mechanisms 214 that allow the patient or healthcare provider to immediately cease operation of the massage system 200. As provided above, the patient can give a command (e.g., Stop!, Ouch!, No!, or other preprogrammed command) that activates the abort mechanism 214. Additionally, or alternatively, the abort mechanism 214 includes a button, lever, or switch that when activated immediately ceases operation of the massage system 200. For example, the patient can be presented with a big red button that when pressed merely ceases operation of the massage system 200.

In some embodiments, when any of the abort mechanisms 214 our activated, the robotic arms 202 and any other component of the massage system 200 within a 1-foot radius of the patient is retracted away from the patient (e.g., to a distance greater than 1 foot, greater than 2 feet, greater than 3 feet, greater than 4 feet, greater than 5 feet, or farther).

It should be appreciated that massage systems of the present disclosure are designed for patient health and well-being and not to injure or otherwise hurt the patient. By ensuring the robotic arms 202 do not exceed a threshold amount of tactile pressure, preferably 10 psi or less, and do not press into the patient beyond a threshold depth, preferably 1 inch or less, the massage system 200 is prevented from operating in a manner that would crush the patient or otherwise injure or maim the patient. Furthermore, the inclusion of abort mechanisms 214 provide additional safety precautions that can prevent injury or discomfort to the patient.

In some embodiments, the massage system 200 additionally includes a mobile device 216. The mobile device 216 can, for example, be a patient device (e.g., a smartphone or tablet) that stores the massage therapy protocol 206, a patient medical history, the patient massage therapy history, a patient preference library, or communicates such information to a computer system 210. In some embodiments, the computer system 210 saves and/or updates one or more of the user massage therapy history, user preference library, or patient medical history to the mobile device 216. This can advantageously enable the patient to have access to their own personal data and preferences and to provide such preferences or personalized massage therapy protocols to similar computing systems that operate an automated massage therapy system (similar to massage system 200, for example).

In some embodiments, the mobile device 216 includes a user interface. The user interface can allow patient to program a personalized massage therapy protocol. This can include, for example, the mobile device presenting a visual representation of a body (e.g., the patient's or a generalized cartoon) onto which the user can indicate therapeutic site(s) for massage therapy. Additionally, the user interface may present and receive information related to a duration, massage modality, and/or a level or amount of pressure to be administered during the massage therapy. The mobile device 216 can be in electrical communication with the computer system 210 or with any of the foregoing components within the massage system 200. The collected data can be compiled by the computer system 210 and/or mobile device 216 into a personalized massage therapy protocol and subsequently implemented by an automated robotic massage therapy system associated with the mobile device (e.g., massage system 200 of FIG. 2). In some embodiments, the personalized massage therapy protocol is saved to the mobile device 216 or to another location (e.g., the cloud) for later reference or use.

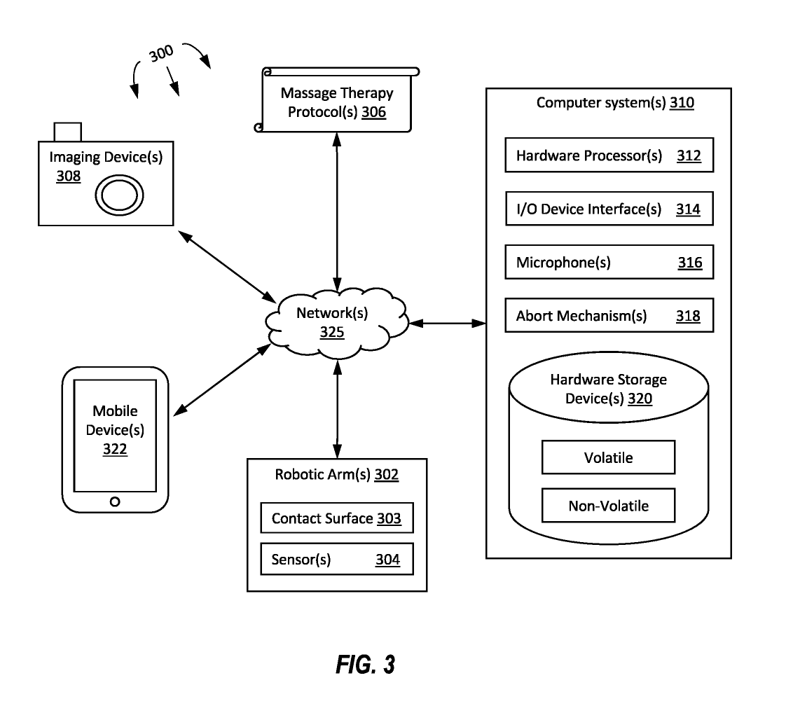

It should be appreciated that in some embodiments, the components of the disclosed massage therapy systems can be communicatively coupled over a network, or the components may communicate through a communication channel directly connecting each component. For example, as shown in FIG. 3, various components of massage system 300 can be communicatively coupled over a network 325. In an exemplary implementation, a massage therapy protocol 306 is received by computer system 310 over network 325. The massage therapy protocol 306 can, for example, be stored on hardware storage device 320 and any of volatile or nonvolatile media. Hardware processors 312 can extract information from the massage therapy protocol 306, such as a therapeutic site and modality of therapy to be implemented. The computer system 310 can communicate with imaging devices 308 to identify a patient's body and to further identify the therapeutic site on the patient's body. In some embodiments, the foregoing can be implanted by an image processor, which can be a hardware processor 312, a software processor, or combinations thereof. After identifying the therapeutic site on the patient's body, the computer system can direct robotic arms 302 to the therapeutic site and implement massage therapy in accordance with massage therapy protocol 306. Sensors 304 associated with robotic arms 302 can communicate an amount of pressure exerted upon contact surfaces of the robotic arms 3022 the computer system 310, which can, when necessary, adjust the positioning of the robotic arms to maintain implementation of the massage therapy, as indicated in the massage therapy protocol 306.

Similar to that described above, a mobile device 322 can be communicatively coupled to a computer system 310 over network 325 and can receive user input through an associated user interface and transmit the user input to the computer system 310. The computer system 310 receives the transmitted user input and can implement a personalized massage therapy protocol in accordance therewith by communicating with and/or controlling one or more of robotic arms 302 and imaging devices 308.

In some embodiments, the massage therapy system includes a bed, table, or other flat surface upon which the patient lies prone. The robotic arms can be positioned above the table and can extend down toward the table to access the patient's body. An imaging device may be stationary above the table or may be positioned on a movable arm to capture three-dimensional images and or provide additional image data to help guide the robotic arms to the therapeutic site. Each of the robotic arms can include any number or type of distal appendage, such as one or a plurality, or a combination of a ball, a roller, a digit, a knob, or other mechanical device used in massage therapy in which may in some embodiments imitate hands, fingers, elbows, forearms, or other appendage or device used or prescribed in massage therapy.

### Methods for Automated Massage Therapy

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

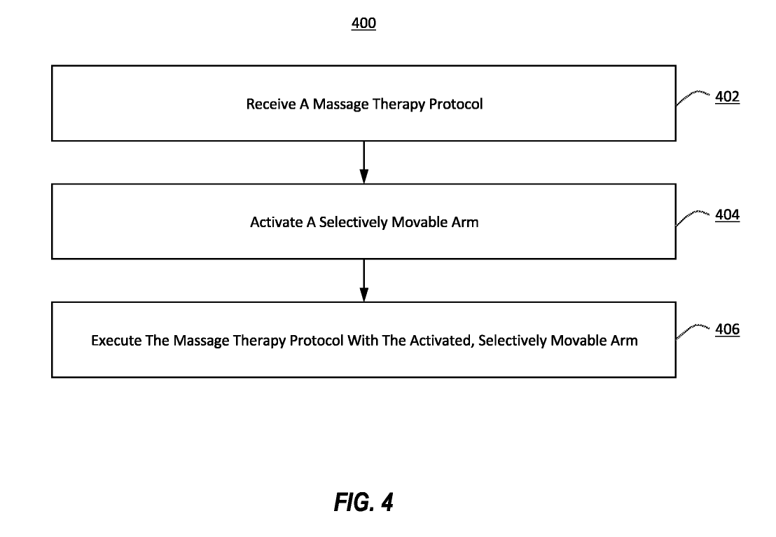

Referring now to FIG. 4, a method 400 is illustrated. The method 400 includes acts for automated massage therapy. The method 400 includes receiving a massage therapy protocol (act 402). As previously noted, the massage therapy protocol can be received over a network or directly from the user at a mobile device (e.g., mobile device 322) or a computing system (e.g., computing system 310). Massage therapy protocol can include a prescribed massage therapy protocol or personalized massage therapy protocol and can be received at the computer system (e.g., computer system 310) or other component of the massage system capable of implementing the massage therapy protocol.

The method 400 further includes activating a selectively movable arm (act 404). The selectively movable arm can be associated with one or more sensors and/or computing system, as described above.

The method 400 further includes executing massage therapy protocol with the activated, selectively movable arm (act 406). Various examples are illustrated above with respect the description related to FIGS. 2 and 3.

The methods may be practiced by a computer system including one or more processors and computer-readable media such as computer memory. In particular, the computer memory may store computer-executable instructions that when executed by one or more processors cause various functions to be performed, such as the acts recited in the embodiments.

### Abbreviated List of Defined Terms

To assist in understanding the scope and content of the foregoing and forthcoming written description and appended claims, a select few terms are defined directly below.

As used herein, the term “healthcare provider” generally refers to any licensed and/or trained person prescribing, administering, or overseeing the diagnosis and/or treatment of an individual or who otherwise tends to the wellness of an individual. This term may, when contextually appropriate, include any licensed medical professional, particularly a chiropractor or a massage therapist, but can extend to other licensed medical professionals, such as a physician (e.g., medical doctor, doctor of osteopathic medicine, etc.), a physician's assistant, a nurse, a nurse's assistant, a veterinarian, etc.

The term “massage therapy,” as used herein is intended to be understood as any manual manipulation of soft body tissues (e.g., muscle, connective tissue, tendons, and/or ligaments) to enhance a person's health and well-being, such as reducing stress and anxiety, relaxing muscles, rehabilitating injuries, reducing pain, and/or otherwise promoting health and wellness. This term generally includes any of the known types—or modalities—of massage therapy methods. Massage therapy, as used herein, includes modalities directed to relaxation massage (commonly known as Swedish massage) often practiced in settings like spas, wellness centers, and resorts. Massage therapy is additionally intended to include modalities directed to rehabilitative massage (commonly known as deep tissue, medical, therapeutic or clinical massage) often practiced in many settings like clinics, hospitals, and chiropractic offices.

As used herein, the term “patient” generally refers to any animal, for example a mammal, under the care of a healthcare provider, as that term is defined herein, with particular reference to humans under the care of a physician, chiropractor, massage therapist, or other relevant professional that oversees or administers massage therapy. For the purpose of the present application, a “patient” may be interchangeable with an “individual” or “person” receiving massage therapy.

Unless defined otherwise, all technical and scientific terms used herein have the same meaning as commonly understood by one of ordinary skill in the art to which the present disclosure pertains.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” “involves,” “contains,” etc.), and similar terms as used herein, including within the claims, shall be inclusive and/or open-ended, shall have the same meaning as the word “comprising” and variants thereof (e.g., “comprise” and “comprises”), and do not exclude additional un-recited elements or method steps, illustratively.

It will be noted that, as used in this specification and the appended claims, the singular forms “a,” “an” and “the” include plural referents unless the context clearly dictates otherwise. Thus, for example, reference to a singular referent (e.g., “widget”) includes one, two, or more referents. Similarly, reference to a plurality of referents should be interpreted as comprising a single referent and/or a plurality of referents unless the content and/or context clearly dictate otherwise. For example, reference to referents in the plural form (e.g., “widgets”) does not necessarily require a plurality of such referents. Instead, it will be appreciated that independent of the inferred number of referents, one or more referents are contemplated herein unless stated otherwise.

As used herein, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure and/or claimed invention.

### CONCLUSION

Any headings used herein are for organizational purposes only and are not meant to be used to limit the scope of the description or the claims.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A robotic system for massage therapy, comprising:
a selectively movable robotic arm having a contact surface; and
a sensor associated with the selectively movable robotic arm, the sensor detecting an amount of tactile pressure exerted upon the contact surface.

2. The robotic system as in claim 1, further comprising an imaging device for imaging at least a portion of a person.

3. The robotic system as in claim 2, wherein the imaging device is selectively movable and configured to capture a three-dimensional image of at least a portion of a person.

4. The robotic system as in claim 2, wherein the imaging device captures one or more of thermal images, ultrasonic images, infrared images, or visible light images.

5. The robotic system as in any one of claim 1, further comprising a computer system associated with one or more of the selectively movable robotic arm and the sensor, the computer system comprising:
one or more processors;
one or more computer-readable hardware storage devices, wherein the one or more computer-readable hardware storage devices comprise computer-executable instructions that when executed by at least one of the one or more processors cause the computer system to perform at least the following:
receive image data from the imaging device; and
identify a therapeutic site based on the image data.

6. The robotic system as in claim 5, wherein when executed, the computer-executable instructions further cause the computer system to position the contact surface of the selectively movable arm at or near the therapeutic site.

7. The robotic system as in claim 5, wherein when executed, the computer-executable instructions further cause the computer system to receive a massage therapy protocol and activate the selectively movable arm to perform the massage therapy protocol.

8. The robotic system as in claim 7, wherein the massage therapy protocol comprises one or more of a location of the therapeutic site, a massage therapy modality to apply at the therapeutic site, a duration of therapy, or one or more amounts of tactile pressure to apply during therapy.

9. The robotic system as in claim 7, wherein when executed, the computer-executable instructions further cause the computer system to perform at least the following:
receive tactile pressure data from the sensor during therapy; and
modify a position of the selectively movable arm to increase or decrease the amount of tactile pressure exerted upon the contact surface in accordance with the massage therapy protocol.

10. The robotic system as in claim 5, wherein when executed, the computer-executable instructions further cause the computer system to perform at least the following:
receive tactile pressure data from the sensor during therapy; and
modify a position of the selectively movable arm to decrease the amount of tactile pressure exerted upon the contact surface in accordance with a maximum tactile pressure threshold.

11. The robotic system as in claim 7, further comprising one or more of a microphone or a user interface for receiving patient commands.

12. The robotic system as in claim 11, wherein when executed, the computer-executable instructions further cause the computer system to perform at least the following:
receive a command from one or more of the microphone or the user interface; and
adjust the massage therapy protocol based on the received command.

13. The robotic system as in claim 12, wherein adjusting the massage therapy protocol includes one or more of increasing or decreasing a duration of at least a portion of the massage therapy protocol, increasing or decreasing the amount of tactile pressure exerted upon the contact surface, changing the massage therapy modality.

14. The robotic system as in claim 1, further comprising an abort mechanism, wherein upon activation of the abort mechanism, the robotic system is retracted to a safety location for a period of time.

15. A computer system for massage therapy, comprising:
one or more processors;
one or more computer-readable hardware storage devices, wherein the one or more computer-readable hardware storage devices comprise computer-executable instructions that when executed by at least one of the one or more processors cause the computer system to perform at least the following:
receive a massage therapy protocol;
receive image data from an imaging device in electrical communication with the computer system;
based on the image data and the massage therapy protocol, identify a therapeutic location; and
activate a selectively movable arm in electrical communication with the computer system to execute at least a portion of the massage therapy protocol, the selectively movable arm having a contact surface and being associated with a sensor for detecting an amount of tactile pressure exerted upon the contact surface.

16. The computer system as in claim 15, wherein the massage therapy protocol is received from a healthcare provider or a patient over a network, from a microphone in electrical communication with the computer system, or through a user interface in electrical communication with the computer system.

17. The computer system as in claim 15, wherein when executed, the computer-executable instructions further cause the computer system to perform at least the following:
receive tactile pressure data from the sensor during execution of the at least a portion of the massage therapy protocol; and
modify the position of the selectively movable arm to increase or decrease the amount of tactile pressure exerted upon the contact surface in accordance with the massage therapy protocol or in accordance with a maximum tactile pressure threshold.

18. The computer system as in claim 16, wherein when executed, the computer-executable instructions further cause the computer system to perform at least the following:
receive a command from one or more of the microphone or the user interface; and
adjust the massage therapy protocol based on the received command.

19. The computer system as in claim 15, wherein when executed, the computer-executable instructions further cause the computer system to perform at least the following:
receive an abort command; and
retract the selectively movable arm to a safety location for a period of time.

20. A method for automated massage therapy, the method comprising:
receiving a massage therapy protocol;
activating a selectively movable arm, the selectively movable arm having a contact surface and being associated with a sensor for detecting an amount of tactile pressure exerted upon the contact surface; and
executing the massage therapy protocol with the activated, selectively movable arm.
